(19) United States
(12) Patent Application Publication
(10) Pub. No.: US 2026/0166535 A1
(43) Pub. Date: Jun. 18, 2026

(54) MTW FRAMEWORK-TYPE MOLECULAR SIEVES, AND METHODS OF MAKING AND USING THE SAME

(71) Applicant: BRASKEM AMERICA, INC., Philadelphia, PA (US)

(72) Inventors: Dan Xie, Pittsburgh, PA (US); Ishant Khurana, Pittsburgh, PA (US); Graham Gregorich, Pittsburgh, PA (US); Richard Opfer, Pittsburgh, PA (US)

(21) Appl. No.: 19/417,768

(22) Filed: Dec. 12, 2025

Related U.S. Application Data
(60) Provisional application No. 63/733,971, filed on Dec. 13, 2024.

(51) Int. Cl. B01J 29/76; B01J 37/04; B01J 37/08; C07C 5/333

(52) U.S. Cl. CPC B01J 29/7669; B01J 37/04; B01J 37/08; C07C 5/333; C07C 2529/76

(57) ABSTRACT

The disclosure provides an MTW framework-type molecular sieve with crystallites having an average size of 70 nm or below. The sieve includes a structure-directing composition comprising 2-pyrrolidone or its N-alkyl derivative and a quaternary ammonium cation. A method for preparing the sieve and its use in producing olefins from alkanes are also described.

### CROSS-REFERENCE TO RELATED APPLICATIONS

This application claims benefit of priority under 35 U.S.C. § 119(e) to U.S. Provisional Patent Application No. 63/733,971, filed on Dec. 13, 2024, which is hereby incorporated by reference in its entirety.

### FIELD OF INVENTION

The present disclosure relates to molecular sieves, and more particularly to novel MTW framework-type molecular sieves, methods of making such molecular sieves, and methods of using such molecular sieves for producing olefins.

### BACKGROUND

Molecular sieves are crystalline materials with well-defined pore structures that can selectively adsorb molecules based on their size and shape. These materials have found widespread use in various industrial applications, including catalysis, gas separation, and purification processes. Among the different types of molecular sieves, zeolites are particularly important due to their unique properties and versatility.

Zeolites are aluminosilicate materials with three-dimensional frameworks composed of SiO₄ and AlO₄ tetrahedra. The arrangement of these tetrahedra creates a network of channels and cavities of molecular dimensions. The presence of aluminum in the framework introduces a negative charge, which is balanced by exchangeable cations. This feature, along with their high surface area and thermal stability, makes zeolites valuable in catalytic applications.

One particular zeolite framework type of interest is the MTW (Mobil TWelve) structure. MTW zeolites are characterized by a one-dimensional pore system with 12-membered ring channels. This unique pore architecture gives MTW zeolites specific catalytic properties that are desirable for certain industrial processes, such as the production of light olefins from alkanes.

The synthesis of zeolites typically involves the use of structure-directing agents (SDAs), which are organic molecules that guide the formation of specific zeolite structures. The choice of SDA, along with other synthesis parameters such as temperature, time, and reactant composition, plays a role in determining the final zeolite structure and properties.

In recent years, there has been growing interest in developing new synthesis methods for zeolites, including MTW-type materials, to improve their properties or to make their production more economical. These efforts have focused on exploring novel SDAs, adjusting synthesis conditions, and investigating the use of different silicon and aluminum sources.

Despite the progress made in zeolite synthesis, there remains a need for improved methods to produce MTW-type zeolites with desirable characteristics, such as smaller crystal sizes or enhanced catalytic performance. Additionally, the development of more efficient and cost-effective synthesis routes continues to be an important goal in the field of zeolite research and development.

### SUMMARY

According to an aspect of the disclosure, an MTW framework-type molecular sieve is provided. The molecular sieve includes a plurality of crystallites of a Mobil TWelve (MTW) framework-type zeolite, wherein the crystallites have an average crystal size of 70 nm or below, as measured by X-ray powder diffraction. The molecular sieve also includes a structure-directing composition (SDC) present within the zeolite's pore structures, wherein the structure-directing composition comprises (1) 2-pyrrolidone or its N-alkyl derivative, and (2) a quaternary ammonium cation represented by N⁺R₁R₂R₃R₄, wherein R₁, R₂, R₃, and R₄ are each independently an alkyl.

According to another aspect of the disclosure, the MTW framework-type molecular sieve includes a plurality of crystallites of a Mobil TWelve (MTW) framework-type zeolite, wherein the crystallites exhibit an X-ray powder diffraction having at least three peaks at the following 2θ diffraction angles: 7.4, 7.6, 8.8, 15.3, 18.8, and 20.0° 2θ=0.2° 2θ. The molecular sieve also includes a structure-directing composition (SDC) present within the zeolite's pore structures, wherein the structure-directing composition comprises (1) 2-pyrrolidone or its N-alkyl derivative, and (2) a quaternary ammonium cation represented by N⁺R₁R₂R₃R₄, wherein R₁, R₂, R₃, and R₄ are each independently an alkyl.

The MTW framework-type molecular sieve may include one or more of the following features. In component (2) of the structure-directing composition, R₁, R₂, R₃, and R₄ may each independently be methyl or ethyl, and R₁, R₂, R₃, and R₄ may be the same, or at least one of R₁, R₂, R₃, and R₄ may be different than the others. The structure-directing composition may comprise (1) N-methyl-2-pyrrolidone and (2) a tetraethylammonium cation (TEA). The zeolite may comprise silicon oxide (SiO₂) and aluminum (Al₂O₃), and the SiO₂:Al₂O₃ molar ratio in the zeolite may be about 20 or greater.

According to another aspect of the disclosure, a method for preparing a molecular sieve having a MTW framework type is provided. The method includes providing a mixture comprising: (1) a source of silicon oxide (SiO₂), (2) a source of aluminum (Al₂O₃), (3) a source of hydroxide ions (OH⁻), (4) a source of alkali or alkaline earth metal ions (M), (5) a structure-directing composition (SDC) comprising (1) 2-pyrrolidone or its N-alkyl derivative, and (2) a quaternary ammonium cation represented by N⁺R₁R₂R₃R₄, wherein R₁, R₂, R₃, and R₄ are each independently an alkyl, and (6) water. The method also includes subjecting the mixture to crystallization conditions sufficient to form a plurality of crystallites of an MTW framework-type zeolite, having the structure-directing composition within the zeolite's pore structures.

According to another aspect of the present disclosure, a process for producing one or more olefins is provided. The process includes reacting an alkane feedstock in the presence of the MTW framework-type molecular sieve to convert the alkane to one or more olefins, via oxidative dehydrogenation reaction. The alkane feedstock may be a C₂-C₄ alkane, and the olefin may be a C₂-C₄ olefin. The reaction may be carried out at a temperature of about 400° C. or below.

### BRIEF DESCRIPTION OF FIGURES

FIG. 1 illustrates an X-ray diffraction pattern of the synthesized MTW framework-type molecular sieve in Example 1.

FIG. 2 depicts a graph comparing olefin formation rates using the disclosed Cu-H-MTW catalyst and a commercial microporous catalyst.

### DETAILED DESCRIPTION

The present disclosure relates to MTW framework-type molecular sieves and their applications. MTW framework-type molecular sieves are a type of solid acid material and classified as a zeolite with a unique crystal structure. In some cases, MTW framework-type molecular sieves may be used as catalysts for a variety of catalytic applications, including the conversion of alkanes to olefins, and petrochemical productions using novel routes to reduce carbon footprint and renewable chemical conversions.

The MTW framework-type molecular sieve of the disclosed invention may comprise a plurality of crystallites of a Mobil TWelve (MTW) framework-type zeolite. In some cases, the crystallites may have an average crystal size of 70 nm or below (such as 65 nm or below, 60 nm or below, 55 nm or below, or 50 nm or below), as measured by X-ray powder diffraction.

The MTW framework-type molecular sieve may include a structure-directing composition (SDC) present within the zeolite's pore structures. In some cases, the structure-directing composition may comprise two main components: (1) 2-pyrrolidone or its N-alkyl derivative, and (2) a quaternary ammonium cation. It has been found that such a combination of organic species unexpectedly produces nanocrystal forms of the product. For instance, smaller crystallites may provide a larger external surface area and shorter diffusion paths for reactants and products, potentially enhancing the overall reaction rate.

The first component of the structure-directing composition may be 2-pyrrolidone or an N-alkyl derivative of 2-pyrrolidone. In some cases, the N-alkyl derivative may be N-methyl-2-pyrrolidone.

The second component of the structure-directing composition may be a quaternary ammonium cation represented by N⁺R₁R₂R₃R₄, wherein R₁, R₂, R₃, and R₄ may each independently be an alkyl group. In some cases, R₁, R₂, R₃, and R₄ may each independently be a methyl or ethyl group. The R groups may all be the same, or at least one of R₁, R₂, R₃, and R₄ may be different than the others.

In some cases, the quaternary ammonium cation may be a tetraethylammonium cation (TEA). The TEA may be present in the form of a salt. Examples of suitable TEA salts may include tetraethylammonium bromide (TEABr), tetraethylammonium chloride (TEACl), tetraethylammonium hydroxide (TEAOH), or a mixture thereof.

A specific example of a structure-directing composition is one that comprises (1) N-methyl-2-pyrrolidone and (2) a tetraethylammonium cation (TEA). In this case, the N-methyl-2-pyrrolidone serves as the N-alkyl derivative of 2-pyrrolidone, while the TEA serves as the quaternary ammonium cation where all four R groups are ethyl.

The presence of this structure-directing composition within the zeolite's pore structures may influence the formation and properties of the MTW framework-type molecular sieve. The specific combination of the 2-pyrrolidone derivative and the quaternary ammonium cation may guide the assembly of the zeolite framework during synthesis, potentially affecting characteristics such as crystal size, pore structure, and catalytic properties.

The MTW framework-type molecular sieve may comprise silicon oxide (SiO₂) and aluminum (Al₂O₃). In some cases, the SiO₂:Al₂O₃ molar ratio in the zeolite may be about 20 or greater. For example, the SiO₂:Al₂O₃ molar ratio may range from about 20 to about 50, or from about 20 to about 40, or from about 25 to about 35.

The specific SiO₂:Al₂O₃ molar ratio may influence various properties of the MTW framework-type molecular sieve, such as its acidity, thermal stability, and catalytic activity. A higher SiO₂:Al₂O₃ ratio may result in a more hydrophobic zeolite with fewer acid sites, which may be advantageous for certain applications.

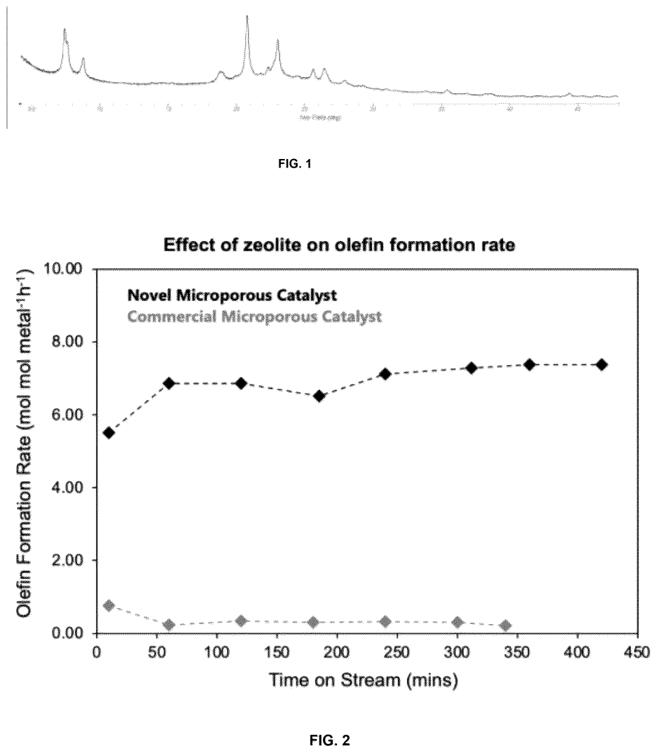

FIG. 1 shows an exemplary X-ray powder diffraction pattern for an MTW framework-type molecular sieve. In one embodiment, the X-ray powder diffraction pattern of the disclosed MTW framework-type molecular sieve exhibits at least three peaks at the following 2θ diffraction angles: 7.4, 7.6, 8.8, 15.3, 18.8, and 20.0° 2θ+0.2° 2θ. In some cases, the X-ray powder diffraction pattern may exhibit at least four, five, or six peaks at these angles.

The X-ray powder diffraction pattern can also be used to determine the crystal size of the MTW framework-type molecular sieves. The Scherrer Equation is used to relate the peak broadening (measured as full width at half-maximum, FWHM) to the average crystallite size. Broader peaks indicates smaller crystals and vice versa. The Scherrer Equation is shown below:

$$D = ( K * λ ) / ( β * cos θ )$$

$$where :$$

$$D = average crystallite size ;$$

$$K = shape factor ;$$

$$λ = X - ray wavelength ;$$

$$β = FWHM of the diffraction peak in radians ;$$

$$θ = Bragg diffraction angle .$$

In some cases, the MTW framework-type molecular sieve may have a specific surface area. For example, the specific surface area may be greater than about 300 cm²/g (such greater than as about 305 cm²/g, or greater than about 310 cm²/g), as measured by Ar adsorption isotherms obtained at 87K on a Surface Area and Porosity Analyzer. For example, the specific surface area may be about 308 cm²/g.

The MTW framework-type molecular sieve may also have a specific micropore volume. The micropore volume may be measured by Ar adsorption isotherms obtained at 87K on a Surface Area and Porosity Analyzer. In some cases, the micropore volume may range from about 0.10 to 0.20 cm³/g (such as 0.14 to 0.20 cm³/g or 0.15 to 0.19 cm³/g). For example, the micropore volume may be about 0.17 cm³/g.

These properties, including the SiO₂:Al₂O₃ molar ratio, surface area, and micropore volume, may contribute to the overall performance of the MTW framework-type molecular sieve in various applications, such as catalytic processes. The specific values of these properties may be tailored to optimize the molecular sieve for particular uses or reactions.

Likewise, the structural characteristics of the MTW framework-type molecular sieve, including the crystallite size, X-ray powder diffraction pattern, specific surface area, and micropore volume, may contribute to its performance in various applications, such as catalytic processes.

The MTW framework-type molecular sieve may be prepared by a method comprising several steps. In some cases, the method may begin with providing a mixture comprising various components. The mixture may include a source of silicon oxide (SiO₂), a source of aluminum (Al₂O₃), a source of hydroxide ions (OH⁻), a source of alkali or alkaline earth metal ions (M), a structure-directing composition (SDC), and water (such as deionized water).

In some cases, the source of silicon oxide may be colloidal silica, precipitated silica, fumed silica, alkali metal silicates, tetraalkyl orthosilicates, or a mixture thereof. The source of aluminum may be hydrated alumina, a water-soluble aluminum salt, or a mixture thereof. In some implementations, the water-soluble aluminum salt may be sodium aluminate, aluminum nitrate, or an aluminum sulfate or its hydrate such as aluminum sulfate hexadecahydrate or aluminum sulfate octadecahydrate.

The structure-directing composition may comprise (1) 2-pyrrolidone or its N-alkyl derivative, such as N-methyl-2-pyrrolidone, and (2) a quaternary ammonium cation represented by N⁺R₁R₂R₃R₄, wherein R₁, R₂, R₃, and R₄ may each independently be an alkyl group, such as methyl or ethyl. The R groups may all be the same, or at least one of R₁, R₂, R₃, and R₄ may be different than the others.

As noted above, the quaternary ammonium cation may be a tetraethylammonium cation (TEA). The TEA may be present in the form of a salt. Examples of suitable TEA salts may include tetraethylammonium bromide (TEABr), tetraethylammonium chloride (TEACl), tetraethylammonium hydroxide (TEAOH), or a mixture thereof.

In some cases, two or more components in the mixture may be provided via a combined source. For example, the source of silicon oxide and source of aluminum may be provided via a combined source selected from an aluminosilicate zeolite, clays or treated clays, and a mixture thereof. In some implementations, the aluminosilicate zeolite may be a zeolite of a FAU framework-type, such as Zeolite Y. The treated clay may be metakaolin.

In some embodiments, the source of silicon oxide is colloidal silica, and the source of aluminum is a water-soluble aluminum salt (e.g., an aluminum sulfate or its hydrate, such as aluminum sulfate hexadecahydrate or aluminum sulfate octadecahydrate); or the source of silicon oxide and source of aluminum are a zeolite of a FAU framework-type (e.g., Zeolite Y).

The components of the mixture may be characterized by specific molar ratios. In some cases, one or more of these molar ratios, or combinations thereof, may be:
SiO₂:Al₂O₃: ≥20 (e.g., 20 to 60, 20 to 50, 20 to 40, or 25 to 35),
SDC:SiO₂: 0.05 to 0.60 (e.g., 0.05 to 0.40, 0.05 to 0.30, 0.10 to 0.30),
M:SiO₂: 0.05 to 0.50 (e.g., 0.05 to 0.30, 0.05 to 0.20, 0.15 to 0.50, 0.15 to 0.30, or 0.15 to 0.20),
OH⁻:SiO₂: 0.10 to 0.50 (e.g., 0.15 to 0.50, or 0.20 to 0.45), and
H₂O:SiO₂: 3 to 60 (e.g., 5 to 60, 5 to 40, 5 to 20, 5 to 10, 10 to 60, 10 to 40, 10 to 20, 15 to 60, or 15 to 40).

In some cases, a seed crystal of molecular sieve having a MTW framework type may be added to the mixture. The seed crystal may be added in an amount of 0.01 to 10,000 ppm by weight of the mixture. In some implementations, the amount may be 100 to 5000 ppm by weight of the mixture.

The mixture may then be subjected to crystallization conditions sufficient to form a plurality of crystallites of an MTW framework-type zeolite, having the structure-directing composition within the zeolite's pore structures. In some cases, the crystallization conditions may comprise heating the mixture at a temperature ranging from about 120° C. to 180° C. For example, the temperature may range from about 140 to about 160° C., or from about 150 to about 160° C. The crystallization may be carried out in an autoclave reactor.

In some implementations, the crystallization may be carried out at 150° C. for 5 days with a tumbling speed of approximately 50 rpm.

After crystallization, the formed crystallites of the MTW framework-type zeolite may undergo post-synthesis treatments. In some cases, the formed crystallites may be calcined at a temperature of about 400° C. or above for a time sufficient to remove at least part of the structure-directing composition. For example, the calcination temperature may be about 450° C. or above, about 500° C. or above, about 550° C. or above, or about 550° C.

In some implementations, the calcination may be carried out at 550° C. to remove organic species.

The method may further comprise replacing the metal ions of the formed crystallites of the MTW framework-type zeolite with one or more cations. In some cases, the cations may be selected from the group consisting of hydrogen, rare earth metals, and Group 2 to 15 metals. This replacement may be carried out via an ion exchange treatment.

In some implementations, the replacing step may comprise subjecting the formed crystallites of the MTW framework-type zeolite to hydrogen ions or a hydrogen precursor to replace the metal ions in the zeolite with hydrogen ions. The hydrogen precursor may be ammonium ions, such as those provided by an ammonium nitrate solution.

In some cases, the formed crystallites may be treated with 0.1 N ammonium nitrate solution at 95° C. for 2 hours. This treatment may be repeated two or three times.

The replacing step may also comprise subjecting the formed crystallites of the MTW framework-type zeolite to rare earth metals or Group 2 to 15 metals, or their precursors, to load these metals to the zeolite. In some implementations, the Group 2 to 15 metals may be transition metals, such as a Group 11 metal like Cu.

In some cases, copper may be loaded via solid-state ion-exchange. This may involve physically mixing a copper nitrate precursor with the proton form of the MTW framework-type zeolite, grinding the mixture, and then heating the mixture to a high temperature under the flow of air.

In one embodiment, this invention relates to a method for preparing a molecular sieve having a MTW framework type, as noted above, in which the source of silicon oxide is colloidal silica, and the source of aluminum is a water-soluble aluminum salt (e.g., an aluminum sulfate or its hydrate such as aluminum sulfate hexadecahydrate or aluminum sulfate octadecahydrate), or the source of silicon oxide and source of aluminum are a zeolite of a FAU framework-type (e.g., Zeolite Y); the structure-directing composition comprises (1) N-methyl-2-pyrrolidone and (2) a tetraethylammonium (TEA) cation (such as a salt of TEA, e.g., tetraethylammonium bromide (TEABr), tetraethylammonium chloride (TEACl), tetraethylammonium hydroxide (TEAOH), or a mixture thereof); the crystallization conditions comprise heating the mixture at a temperature ranging from about 120° C. to 180° C. (such as from about 140 to about 160° C., or from about 150 to about 160° C.), optionally in an autoclave reactor; and the method further comprises: calcining the formed crystallites of a MOR framework-type zeolite at a temperature of about 400° C. or above (such as about 450° C. or above, about 500° C. or above, about 550° C. or above, or about 550° C.) for a time sufficient to remove at least part of the structure-directing composition; subjecting the formed crystallites of a MTW framework-type zeolite to hydrogen ions or a hydrogen precursor (such as ammonium ions, e.g., ammonium nitrate solution) to replace the metal ions in the zeolite with hydrogen ions; and subjecting the formed crystallites of a MTW framework-type zeolite to rare earth metals or Group 2 to 15 metals (e.g., transition metals such as Group 11 metal, e.g., Cu), or their precursors, to load the rare earth metals or Group 2 to 15 metals (e.g., transition metals such as Group 11 metal, e.g., Cu) to the zeolite.

In another embodiment, the invention relates to MTW framework-type molecular sieves prepared according any of the above-disclosed methods.

The specific combination of source materials, structure-directing composition, crystallization conditions, and post-treatment steps may result in an MTW framework-type molecular sieve with properties suitable for various applications, such as catalytic processes.

The MTW framework-type molecular sieve may be utilized in various catalytic applications, particularly in the production of olefins from alkane feedstocks via oxidative dehydrogenation reactions. In some cases, the MTW framework-type molecular sieve may serve as a catalyst for converting alkanes to olefins.

Accordingly, some embodiments of this invention relate to a process for producing one or more olefins, comprising reacting an alkane feedstock in the presence of the above-described MTW framework-type molecular sieve, to convert the alkane to one or more olefins, via oxidative dehydrogenation reaction. The alkane feedstock may be a C₂-C₄ alkane, and the resulting olefin product may correspondingly be a C₂-C₄ olefin. For example, the process may involve converting ethane to ethylene, propane to propylene, or butane to butylene.

As an example, when the alkane feedstock is ethane, and the olefin product is the ethane feed concentration may be about 35% in nitrogen, and the total feed rate for the ethane dehydrogenation reaction may be about 200 ml/min. These conditions may be varied according to the feedstock, and various other factors appreciated by one skilled in the art.

The MTW framework-type molecular sieve may exhibit high selectivity and activity for these alkane-to-olefin conversions. In some cases, the molecular sieve may maintain its catalytic performance over extended periods of time, as suggested by the sustained activity shown in FIG. 2 for ethane dehydrogenation.

The reaction may be carried out at a temperature of about 400° C. or below. In some cases, the reaction temperature may be about 350° C. or below. These lower reaction temperatures may offer advantages in terms of energy efficiency and catalyst stability.

The specific properties of the MTW framework-type molecular sieve, such as its pore structure, surface area, and acidity, may contribute to its effectiveness in these catalytic applications. The molecular sieve may provide active sites for the oxidative dehydrogenation reactions while minimizing undesired side reactions.

In some implementations, the reaction conditions, such as temperature, feed concentration, and flow rate, may be optimized for each specific alkane feedstock to maximize olefin yield and selectivity. The ability to operate at lower temperatures, such as 350° C. or below, may offer advantages in terms of energy efficiency and catalyst longevity, as noted above.

The MTW framework-type molecular sieve may interact with alkane feedstocks to catalyze the production of olefins through oxidative dehydrogenation reactions. The structure and composition of the molecular sieve may contribute to its catalytic performance in these reactions.

In some cases, the crystalline structure of the MTW framework-type zeolite may provide a arrangement of pores and channels that allow for the selective adsorption and reaction of alkane molecules. The pore structure may influence the diffusion of reactants and products, potentially affecting the overall reaction kinetics and product selectivity.

The structure-directing composition, comprising 2-pyrrolidone or its N-alkyl derivative and a quaternary ammonium cation, may play a role in shaping the molecular sieve's structure during synthesis. This composition may influence the formation of specific pore sizes and arrangements, which may in turn affect the catalytic properties of the resulting molecular sieve.

In some cases, the presence of the structure-directing composition within the zeolite's pore structures may create specific active sites for the oxidative dehydrogenation reactions. These active sites may facilitate the breaking of C—H bonds in alkane molecules and the formation of C═C bonds to produce olefins.

The SiO₂:Al₂O₃ molar ratio in the zeolite may affect its acidity and hydrophobicity. A higher ratio may result in fewer acid sites and a more hydrophobic surface, which may influence the adsorption and reaction of alkane molecules on the catalyst surface.

In some cases, the surface area and micropore volume of the MTW framework-type molecular sieve may contribute to its catalytic performance. A larger surface area may provide more active sites for the reaction, while the micropore volume may affect the accessibility of these sites to reactant molecules.

The crystallite size of the MTW framework-type zeolite may also play a role in its catalytic activity. Smaller crystallites may provide a larger external surface area and shorter diffusion paths for reactants and products, potentially enhancing the overall reaction rate.

In some cases, the metal ions present in the molecular sieve, whether from the original synthesis or introduced through post-synthesis treatments, may serve as active sites for the oxidative dehydrogenation reactions. These metal ions may facilitate the activation of oxygen and the abstraction of hydrogen from alkane molecules.

The combination of these structural and compositional features may result in a molecular sieve with high selectivity and activity for alkane-to-olefin conversions. The specific arrangement of pores, active sites, and metal ions may allow for efficient conversion of alkanes to olefins while minimizing undesired side reactions.

In some cases, the stability of the MTW framework-type molecular sieve under reaction conditions may contribute to its sustained catalytic performance over time. The robust structure may resist degradation at elevated temperatures and in the presence of reactant and product molecules.

The interaction between the alkane feedstock and the molecular sieve may involve several steps. The alkane molecules may first adsorb onto the surface or within the pores of the molecular sieve. The adsorbed alkanes may then interact with active sites, potentially including metal ions or acid sites, which may facilitate the breaking of C—H bonds. Simultaneously, oxygen may be activated on the catalyst surface. The activated oxygen species may then abstract hydrogen from the alkane molecules, leading to the formation of olefin products.

In some cases, the specific pore structure of the MTW framework-type molecular sieve may influence the product distribution by favoring the formation of certain olefins based on shape selectivity. The pore dimensions may allow for the preferential diffusion of specific product molecules, potentially enhancing the selectivity towards desired olefins.

The overall catalytic performance of the MTW framework-type molecular sieve in alkane-to-olefin conversions may result from the interplay of these various structural, compositional, and mechanistic factors. The specific combination of features in the molecular sieve may provide a catalytic environment that enables efficient and selective olefin production under relatively mild conditions.

### EXAMPLES

### Example 1: Synthesis of MTW Type Framework Molecular Sieve

0.97 grams of deionized water, 0.47 grams of 50 wt % NaOH solution, 2.76 grams of Tetraethylammonium hydroxide solution (Sigma-Aldrich), 1.16 grams of N-Methyl-2-pyrrolidone (Sigma-Aldrich), 0.39 grams of aluminum sulfate octadecahydrate (SigmaAldrich), and 4.69 grams of Ludox® HS-30 colloidal silica were mixed together in a Teflon liner. The gel was stirred until it became homogenous. The liner was then capped and placed within a Parr Steel autoclave reactor. The autoclave was then put in an oven heated at 150° C. for 5 days with tumbling speed of ˜50 rpm. The solid products were recovered from the cooled reactor by centrifugation, washed with deionized water and dried at 95° C.

FIG. 1 shows the powder X-ray diffraction (XRD) patterns of the as-synthesized aluminosilicate product of Example 1. As depicted in FIG. 1, the powder XRD pattern of the as-synthesized product showed the typical phase of MTW topology and also indicated decreased crystal size as inferred from the peak broadening in the XRD pattern.

### Example 2: Synthesis of MTW Type Framework Molecular Sieve

0.55 grams of deionized water, 0.28 grams of 50 wt % NaOH solution, 3.45 grams of Tetraethylammonium hydroxide solution (Sigma-Aldrich), 1.16 grams of N-Methyl-2-pyrrolidone (Sigma-Aldrich), 0.39 grams of aluminum sulfate octadecahydrate (SigmaAldrich), and 4.69 grams of Ludox® HS-30 colloidal silica were mixed together in a Teflon liner.

The gel was stirred until it became homogenous. The liner was then capped and placed within a Parr Steel autoclave reactor. The autoclave was then put in an oven heated at 150° C. for 5 days with tumbling speed of ˜50 rpm. The solid products were recovered from the cooled reactor by centrifugation, washed with deionized water and dried at 95° C.

The resulting product was identified by powder XRD as a pure MTW type molecular sieve. The crystal sizes of the product were about the same as the product of Example 1.

### Example 3: Synthesis of MTW Type Framework Molecular Sieve

5.22 grams of deionized water, 0.29 grams of 50 wt % NaOH solution, 2.14 grams of Tetraethylammonium hydroxide solution (Sigma-Aldrich), 1.20 grams of N-Methyl-2-pyrrolidone (Sigma-Aldrich), and 1.50 grams of CBV760 Y-zeolite powder (Zeolyst International, SiO₂/Al₂O₃ mole ratio=60) were mixed together in a Teflon liner. The gel was stirred until it became homogenous. The liner was then capped and placed within a Parr Steel autoclave reactor. The autoclave was then put in an oven heated at 160° C. for 3 days with tumbling speed of ˜50 rpm. The solid products were recovered from the cooled reactor by centrifugation, washed with deionized water and dried at 95° C.

The resulting product was identified by powder XRD as a pure MTW type molecular sieve. The crystal sizes of the product were slightly smaller compared to the product of Example 1.

### Example 4: Converting the Products from as-Synthesized Form to Proton Form

The as-synthesized molecular sieve product of Example 1 was calcined inside a muffle furnace under a flow of air heated to 550° C. at a rate of 2° C./min and held at 550° C. for 5 hours, cooled and then analyzed by powder XRD. The powder XRD data indicated that the material remains stable after calcination to remove the organic species.

After calcination, the products were treated with 0.1 N ammonium nitrate solution at 95° C. for 2 hours. The solution was cooled, decanted, dried at 95 C and this process was repeated three times. The powders were then calcined again in a muffle furnace in the presence of air, heated to 400° C. at a rate of 2° C./min and held for 3 hours to convert the zeolite sample from ammonium form to proton form.

### Example 5: Metal Loading to Molecular Sieve

For the incumbent (conventional) reference molecular sieve catalyst, Cu was incorporated on commercial MOR type framework molecular sieve (CBV21A, NH4-MOR, SAR=20, Zeolyst) via solid-state ion-exchange, wherein 0.08 g of copper nitrate precursor was physically mixed with 5.92 g of proton form of MOR molecular sieve and grounded in a mortar and pestle for about 15 mins or till the solid mixture turns light green or seems homogenous. This solid mixture was then heated to 873 K (0.167 K s⁻¹) under the flow of air in a horizontal tube furnace and held for 6 h before cooling to room temperature.

For the MTW type molecular sieve obtained from Example 4, Cu was supported on the novel product using the same solid-state ion-exchange procedure.

### Example 6: Catalyst Characterization

Surface area and micropore volumes of the catalysts were determined from Ar adsorption isotherms measured at 87 K on a Micromeritics ASAP 2020 Surface Area and Porosity

Analyzer. 0.03-0.05 g of pelleted and sieved sample (nominal diameter between 180-250 μm) were degassed by heating to 120° C. (100 C/min) under vacuum (<5 μmHg) for 2 hours, and then further heating to 350° C. (100 C/min) under vacuum (<5 μmHg) and holding for 9 hours. Volumetric gas adsorption within micropores (cm³ g⁻¹ at STP) was estimated from analysis of semi-log derivative plots of the adsorption isotherm (∂(Vads)/∂(ln(P/PO)) vs. ln(P/PO)) to identify the micropore filling transition (first maximum) and then the end of micropore filling (subsequent minimum). Micropore volumes (cm³ g⁻¹) were obtained by converting standard gas adsorption volumes (cm³ gcat⁻¹ at STP) to liquid volumes using a density conversion factor assuming the liquid density of Ar at −186° C. Surface area and micropore volumes of the catalyst is shown in Table 1 and is in line with what is expected for conventional zeolite molecular sieves.

TABLE 1
Surface area and micropore volume measured on exemplified
zeolite molecular sieve-based catalysts

| | Surface Area | Micropore Volume |
|---|---|---|
| Sample | (cm² g⁻¹) | (cm³ g⁻¹) |
| MTW molecular sieve-based | 308 | 0.17 |
| catalyst of Example 6 | | |

### Example 7: Catalytic Evaluation—Selective Production of Ethylene from Ethane Reaction at Low Temperature

Ethane was fed onto a bed of transition metal incorporated microporous mixed metal oxide (0.2 g) contained in a lab-scale plug flow reactor. The catalyst bed was maintained at temperature of 350° C. Ethane feed concentration was about 35% in nitrogen and total feed rate was about 200 ml/min.

The novel Cu-H-MTW catalyst exhibited higher and more sustained activity at 100% selectivity vs. the incumbent (conventional) Cu-H-MOR catalyst. FIG. 2 illustrates the results comparing the ethylene formation rate as a function of time-on-stream at 350° C. on both the incumbent and the exemplified MTW framework-type molecular sieve catalysts.

FIG. 2 illustrates the performance of the MTW framework-type molecular sieve in the conversion of ethane to ethylene. The graph shows the ethylene formation rate as a function of time-on-stream at 350° C. for both the exemplified MTW framework-type molecular sieve catalyst and a commercial microporous catalyst. As depicted in FIG. 2, the exemplified MTW framework-type molecular sieve catalyst demonstrates a significantly higher and more sustained ethylene formation rate compared to the commercial catalyst.

1. An MTW framework-type molecular sieve, comprising:
a plurality of crystallites of a Mobil TWelve (MTW) framework-type zeolite, wherein the crystallites have an average crystal size of 70 nm or below, as measured by X-ray powder diffraction; and
a structure-directing composition (SDC) present within the zeolite's pore structures, wherein the structure-directing composition comprises (1) 2-pyrrolidone or its N-alkyl derivative, and (2) a quaternary ammonium cation represented by N⁺R₁R₂R₃R₄, wherein R₁, R₂, R₃, and R₄ are each independently an alkyl.

2. An MTW framework-type molecular sieve, comprising:
a plurality of crystallites of a Mobil TWelve (MTW) framework-type zeolite, wherein the crystallites exhibit an X-ray powder diffraction having at least three peaks at the following 2θ diffraction angles: 7.4, 7.6, 8.8, 15.3, 18.8, and 20.0° 2θ+0.2°2θ; and
a structure-directing composition (SDC) present within the zeolite's pore structures, wherein the structure-directing composition comprises (1) 2-pyrrolidone or its N-alkyl derivative, and (2) a quaternary ammonium cation represented by N⁺R₁R₂R₃R₄, wherein R₁, R₂, R₃, and R₄ are each independently an alkyl.

3. The MTW framework-type molecular sieve of claim 1, wherein in component (2) of the structure-directing composition, R₁, R₂, R₃, and R₄ are each independently methyl or ethyl; and wherein R₁, R₂, R₃, and R₄ are the same, or at least one of R₁, R₂, R₃, and R₄ is different than the others.

4. The MTW framework-type molecular sieve of claim 3, wherein the structure-directing composition comprises (1) N-methyl-2-pyrrolidone and (2) a tetraethylammonium cation (TEA).

5. The MTW framework-type molecular sieve of claim 1, wherein the zeolite comprises silicon oxide (SiO₂) and aluminum (Al₂O₃), and the SiO₂:Al₂O₃ molar ratio in the zeolite is about 20 or greater.

6. A method for preparing a molecular sieve having a MTW framework type, comprising:
providing a mixture comprising:
i. a source of silicon oxide (SiO₂),
ii. a source of aluminum (Al₂O₃),
iii. a source of hydroxide ions (OH⁻)
iv. a source of alkali or alkaline earth metal ions (M),
v. a structure-directing composition (SDC) comprising (1) 2-pyrrolidone or its N-alkyl derivative, and (2) a quaternary ammonium cation represented by N⁺R₁R₂R₃R₄, wherein R₁, R₂, R₃, and R₄ are each independently an alkyl, and
vi. water; and
subjecting the mixture to crystallization conditions sufficient to form a plurality of crystallites of an MTW framework-type zeolite, having the structure-directing composition within the zeolite's pore structures.

7. The method of claim 6, further comprising a seed crystal of molecular sieve having a MTW framework type, optionally in an amount of 0.01 to 10,000 ppm by weight of the mixture.

8. The method of claim 6, wherein in component (2) of the structure-directing composition, R₁, R₂, R₃, and R₄ are each independently methyl or ethyl; and wherein R₁, R₂, R₃, and R₄ are the same, or at least one of R₁, R₂, R₃, and R₄ is different than the others.

9. The method of claim 1, wherein the structure-directing composition comprises (1) N-methyl-2-pyrrolidone and (2) a tetraethylammonium cation (TEA).

10. The method of claim 6, wherein the components of the mixture are characterized by the following molar ratios:
SiO₂:Al₂O₃: ≥20,
SDC:SiO₂: 0.05 to 0.60,
M:SiO₂: 0.05 to 0.50,
OH⁻:SiO₂: 0.10 to 0.50, and
H₂O:SiO₂: 3 to 60.

11. The method of claim 6, wherein the source of silicon oxide is colloidal silica, precipitated silica, fumed silica, precipitated silica, alkali metal silicates, tetraalkyl orthosilicates, or a mixture thereof.

12. The method of claim 6, wherein the source of aluminum is hydrated alumina, a water-soluble aluminum salt, or a mixture thereof.

13. (canceled)

14. The method of claim 13, wherein the source of silicon oxide and source of aluminum are provided via a combined source selected from an aluminosilicate zeolite, clays or treated clays, and a mixture thereof.

15. The method of claim 6, wherein:
the source of silicon oxide is colloidal silica, and the source of aluminum is a water-soluble aluminum salt; or
the source of silicon oxide and source of aluminum are a zeolite of a FAU framework-type.

16. (canceled)

17. The method of claim 6, further comprising at least one of:
calcining the formed crystallites of a MTW framework-type zeolite at a temperature of about 400° C. or above for a time sufficient to remove at least part of the structure-directing composition; and
replacing the metal ions of the formed crystallites of a MTW framework-type zeolite with one or more cations selected from the group consisting of hydrogen, rare earth metals, and Group 2 to 15 metals.

18-20. (canceled)

21. The method of claim 6, wherein:
the source of silicon oxide is colloidal silica, and the source of aluminum is a water-soluble aluminum salt, or the source of silicon oxide and source of aluminum are a zeolite of a FAU framework-type;
the structure-directing composition comprises a tetraethylammonium cation (TEA) and N-methyl-2-pyrrolidone;
the crystallization conditions comprise heating the mixture at a temperature ranging from about 120° C. to 180° C.; and
the method further comprises:
calcining the formed crystallites of a MTW framework-type zeolite at a temperature of about 400° C. or above for a time sufficient to remove at least part of the structure-directing composition;
subjecting the formed crystallites of a MTW framework-type zeolite to hydrogen ions or a hydrogen precursor to replace the metal ions in the zeolite with hydrogen ions; and
subjecting the formed crystallites of a MTW framework-type zeolite to rare earth metals or Group 2 to 15 metals, or their precursors, to load the rare earth metals or Group 2 to 15 metals to the zeolite.

22. A MTW framework-type molecular sieve prepared according claim 6.

23. The MTW framework-type molecular sieve of claim 1, having a surface area of greater than about 300 cm⁻²/g, measured by Ar adsorption isotherms obtained at 87K on a Surface Area and Porosity Analyzer.

24. The MTW framework-type molecular sieve of claim 1, having a micropore volume ranging from about 0.10 to 0.20 cm⁻³/g, measured by Ar adsorption isotherms obtained at 87K on a Surface Area and Porosity Analyzer.

25. A process for producing one or more olefins, comprising:
reacting an alkane feedstock in the presence of the MTW framework-type molecular sieve of claim 1, to convert the alkane to one or more olefins, via oxidative dehydrogenation reaction.

26-28. (canceled)
